(19) United States
(12) Patent Application Publication
(10) Pub. No.: US 2022/0331463 A1
(43) Pub. Date: Oct. 20, 2022

(54) EXPANDABLE DISINFECTING DEVICE

(71) Applicant: OXTI CORPORATION, New Taipei City (TW)

(72) Inventor: Chih-Feng Ho, New Taipei City (TW)

(21) Appl. No.: 17/232,158

(22) Filed: Apr. 16, 2021

(51) Int. Cl. A61L 2/10; A61L 2/26

(52) U.S. Cl. CPC A61L 2/10; A61L 2/26; A61L 2202/11; A61L 2202/14

(57) ABSTRACT

An expandable disinfecting device includes a main body and at least one disinfecting lamp. The main body includes a U-shaped base board that is formed with an opening and side boards extending from two sides of the U-shaped base board. The at least one disinfecting lamp is disposed on the side boards of the main body. The disinfecting lamp includes a light emission unit, a detection unit, a control unit, and a power source unit. A door handle is provided as an example for illustration. The main body is attached to a latch of the door handle by means of a double-sided adhesive tape or coating of adhesives. The disinfecting lamp provided on the side boards emit light to irradiate and disinfect the door handle. The detection unit may detect a person approaching the door handle and notifies the control unit to shut down light emitting from the disinfecting lamp.

### TECHNICAL FIELD OF THE INVENTION

The present invention relates to a disinfecting device, and more particularly to an expandable disinfecting device that includes at least one disinfecting lamp.

### DESCRIPTION OF THE PRIOR ART

Infectious diseases have changed human history for many times before. The infectious diseases are one of the causes that result in the greatest number of deaths in the world. With the change of human living fashion, the global weather change causes diversification of infection routes and infection areas of the infectious diseases. Viruses and pathogenic microorganisms are constantly adapting themselves to the environment and changing so that all sorts of emerging infectious diseases and re-emerging infectious diseases occur constantly, such as measles, smallpox, malaria, and flu of the early days and SARS, H1N1, MERS-COV, and COVID-19 of the recent days. The infection routes of such infectious diseases are primarily contact with secretions and droplet transmission.

Viruses may get attached to a door handle or door knob through coughing or sneezing of a patient or direct contact with respiratory tract secretions. The door handle is a highly transmitting route of infection for viruses. Prior art techniques are known, such as Chinese Patent issued as CN210563796U and disclosing a disinfecting door handle, which includes a door hand and also includes a disinfectant liquid storage box. The disinfectant liquid storage box is provided with a material dispensing opening in a bottom thereof. A spraying tube is connected to the material dispensing opening and a free end of the spraying tube is connected to a spraying nozzle. The spraying nozzle is located exactly above the door handle such that a disinfectant liquid stored in the disinfectant liquid storage box may be sprayed, by way of the spraying tube and the spraying nozzle, to the handle.

However, the disinfectant liquid is corrosive and may be irritating to human skin, and exhibits an effect of bleaching and gives off irritating smells, making it harmful to the human body. In addition, in order to achieve disinfection of both inside and outside door handles, there must be an additional disinfectant liquid storage box arranged indoors. This inevitably increases the cost and requires an additional space for the arrangement, and also, expandability of the device is poor.

### SUMMARY OF THE INVENTION

To achieve the above objective, the present invention provides an expandable disinfecting device, which comprises: a main body and at least one disinfecting lamp. The main body comprises a U-shaped base board. The U-shaped base board is formed with an opening. The U-shaped base board is extended from each of two sides thereof to form a side board. The at least one disinfecting lamp is provided on the side boards on the two sides of the main body. The disinfecting lamp comprises a light emission unit, a detection unit, a control unit, and a power source unit.

Preferably, the side boards are provided with a curved edge.

Preferably, the side boards are provided with a power accommodation trough.

Preferably, the disinfecting lamp comprises a receiver unit, and the receiver unit is a wireless signal receiver.

Preferably, the side boards on the two sides of the U-shaped base board are arranged parallel.

A door handle is taken as an example for illustration. The main body is attached to or combined with a latch of the door handle through application of a double-sided adhesive tape or coating of an adhesive, and then, the disinfecting lamp on the side boards may be put into operation to emit light irradiating the door handle for disinfection. The detection unit is operable to detect an area around the door handle, and when it is detected that a human body is approaching, the control unit controls the disinfecting lamp to stop emitting light. In addition, the main body is also provided with a power accommodation trough for receiving and holding therein a power source unit. The disinfecting lamp may further comprise a receiver unit that functions to receive a wireless signal, such that the control unit may respond to the wireless signal to control the light emission unit to emit light.

### BRIEF DESCRIPTION OF THE DRAWINGS

FIG. 1 is a schematic view showing an expandable disinfecting device according to the present invention.

FIG. 2 is a block diagram of a disinfecting lamp.

FIG. 3 is a schematic view illustrating applying a double-sided adhesive tape or coating an adhesive to a latch of a door handle in advance.

FIG. 4 is a schematic view illustrating a U-shaped base board attached to the latch by fitting an opening thereof over the latch.

FIG. 5 is a schematic view illustrating a power accommodation trough formed in a side board.

FIG. 6 is a schematic view illustrating light emitting from a disinfecting lamp to disinfect the door handle.

FIG. 7 is a schematic view illustrating the disinfecting lamp stopping emission of light when a person is approaching.

### DETAILED DESCRIPTION OF THE PREFERRED EMBODIMENT

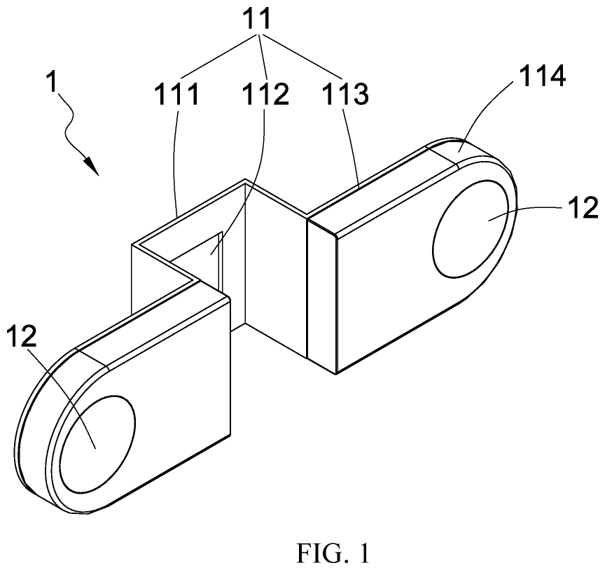

Referring to FIG. 1, the present invention provides an expandable disinfecting device 1, which comprises a main body 11 and at least one disinfecting lamp 12.

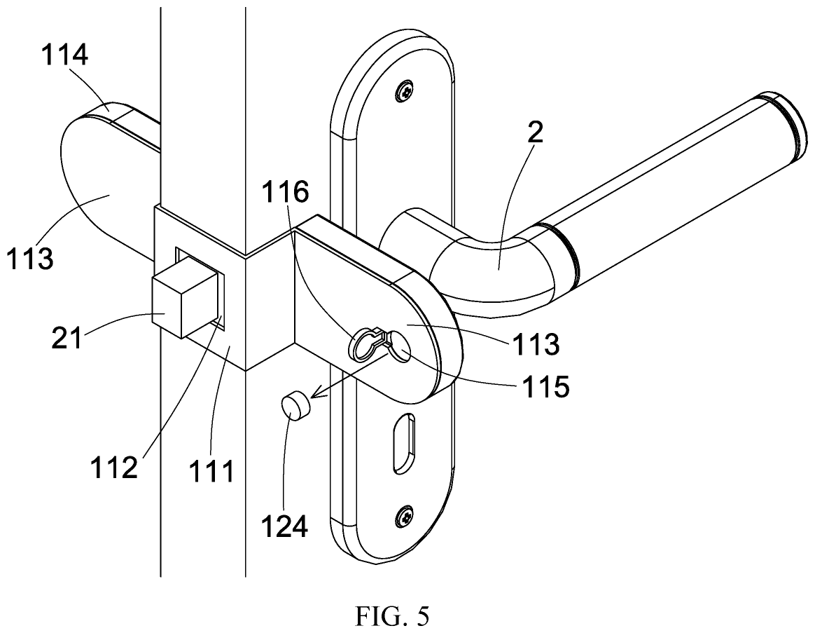

The main body 11 comprises a U-shaped base board 111. The U-shaped base board 111 is formed with an opening 112. The U-shaped base board 111 is extended from each of two sides thereof to form a side board 113. The side boards 113 on the two sides of the U-shaped base board 111 are arranged parallel. The side boards 113 may be provided with a curved edge 114 to prevent cutting or hitting caused by a sharp corner. The side boards 113 may be provided with a power accommodation trough 115 (see FIG. 5). The power accommodation trough 115 is provided with a protective cover 116 (see FIG. 5).

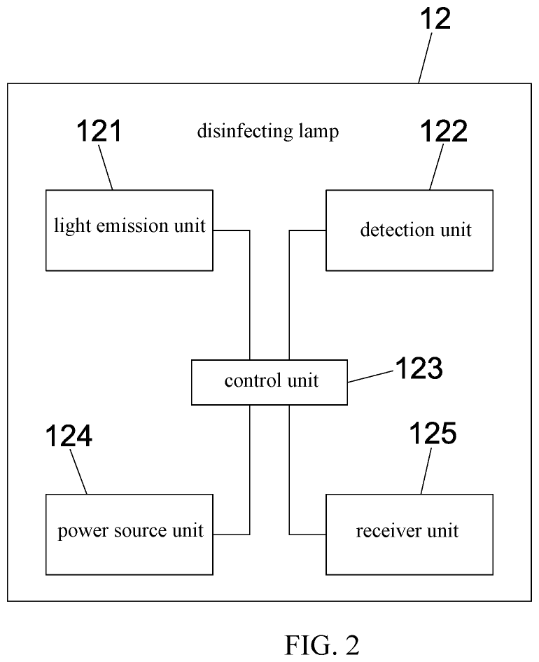

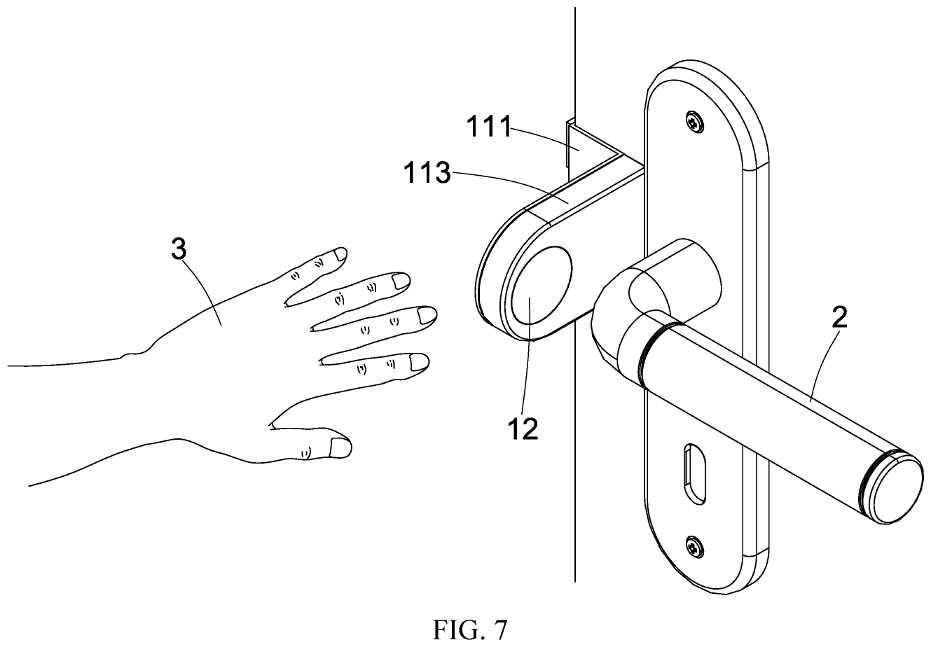

Referring to FIG. 2, the at least one disinfecting lamp 12 is provided on the side boards 113 on the two sides of the main body 11. The disinfecting lamp 12 comprises a light emission unit 121, a detection unit 122, a control unit 123, a power source unit 124, and a receiver unit 125. The at least one disinfecting lamp 12 may include one or multiple, preferably two, such disinfecting lamp of which the number is determined according to requirement. The disinfecting lamp 12 emits light for disinfection. The light may be for example ultraviolet light. The light emission unit 121 is a light source of the disinfecting lamp 12 to give off the light. The light emission unit 121 can be for example a UV-B ultraviolet lamp, a UV-C ultraviolet lamp, a mercury lamp, a metal halide lamp, or a xenon lamp, but not limited thereto. The detection unit 122 functions to detect a human body 3 (see FIG. 7). The detection unit 122 can be an infrared sensor unit, a photosensor unit, an ultrasonic sensor unit, or a thermal sensor unit. The control unit 123 controls operation of the light emission unit 121. The power source unit 124 supplies electrical power. The power source unit 124 can be for example an electrical cell. The power source unit 124 is disposed in the power accommodation trough 115 of the side boards 113 (see FIG. 5). The receiver unit 125 functions to receive a wireless signal. The receiver unit 125 can be for example a wireless signal receiver, such that the control unit 123 may control the light emission unit 121 to generate light according to the wireless signal. For example, a smart phone or a remote control may be used to transmit the wireless signal to the receiver unit 125.

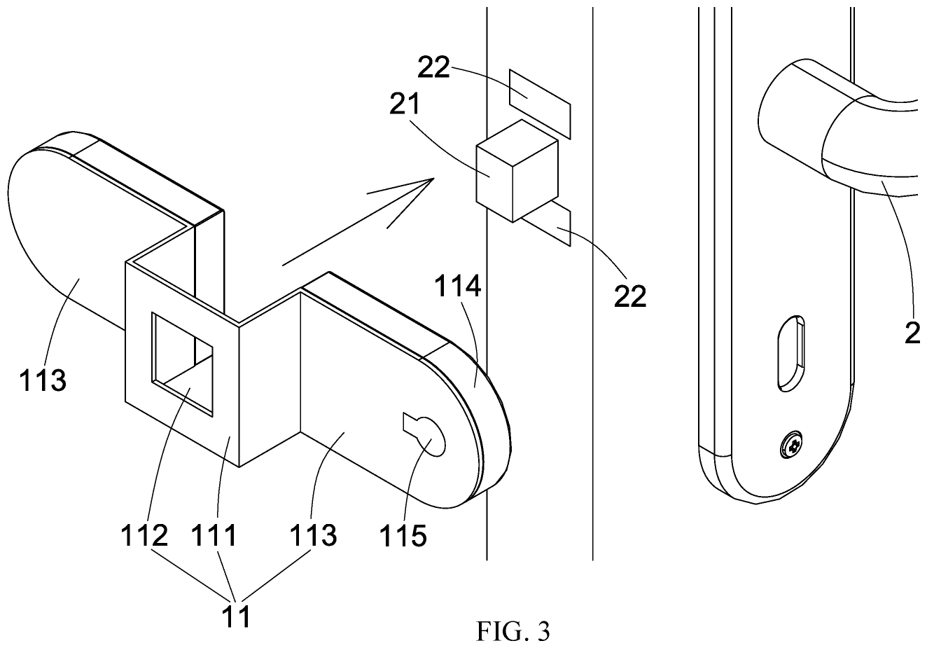

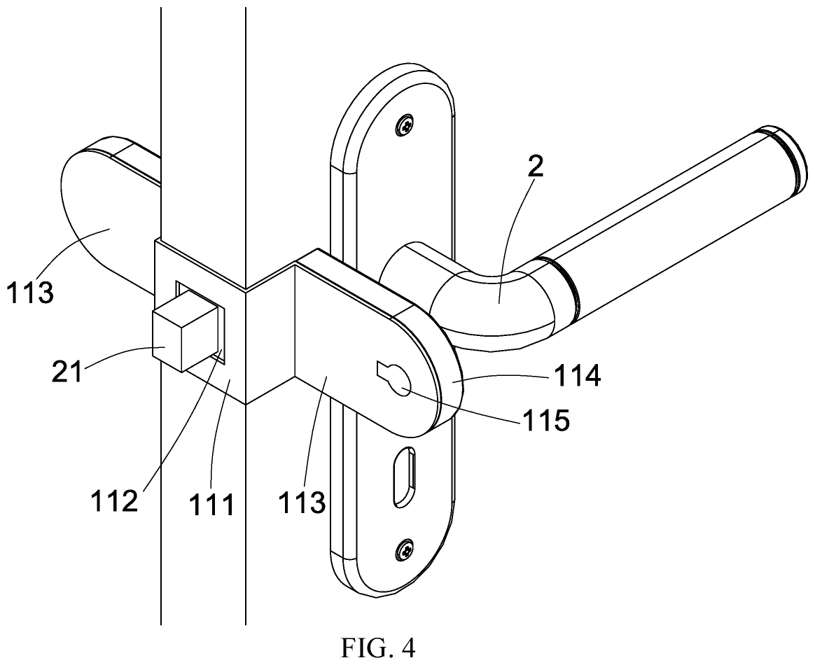

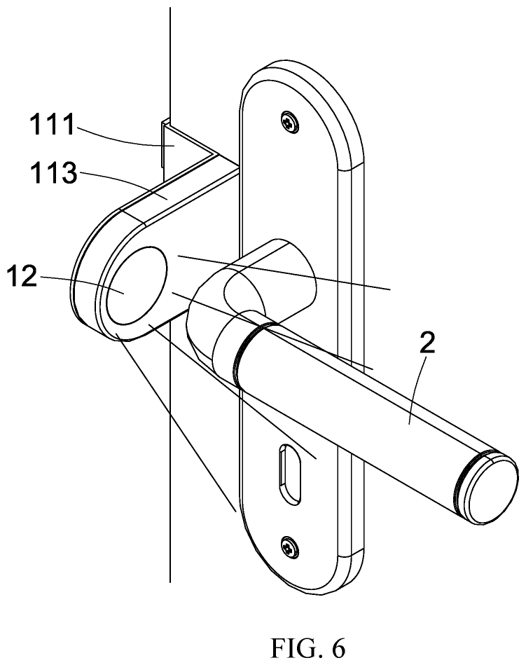

Referring to FIGS. 3 and 4, a door handle 2 will be taken as an example of illustration below. A double-sided adhesive tape 22 or coating of adhesive is applied to an area around a latch 21 in advance. The main body 11 is then set to have the latch 21 received through the opening 112 of the U-shaped base board 111 so as to be attached to or combined with the latch 21 of the door handle 2 by means of the double-sided adhesive tape 22 or the coated adhesive. Referring to FIG. 6, the disinfecting lamp 12 provided on the side boards 113 may then put into operation to emit light to irradiate the door handle 2 to proceed with disinfection. Referring to FIGS. 2 and 7, the detection unit 122 may detect an area around the door handle 2, such that when it is detected that a human body 3 is approaching, the detection unit 122 notifies the control unit 123, and the control unit 123 controls the disinfecting lamp 12 to stop emitting light in order to prevent hurting the human body 3.

In summary, the expandable disinfecting device 1 comprises at least one disinfecting lamp 12 provided on the side boards 113 on two sides of the U-shaped base board 111 and the disinfecting lamp 12 is operable to irradiate door handles 2 on both indoor side and outdoor side so as to ensure security indoors and outdoors with an effect of disinfection that is better than a disinfectant agent. Further, the disinfecting lamp 12 can be expanded without occupying additional space and the only cost is the expense of the disinfecting lamp 12. Thus, the cost is not increased greatly. Further, selection can be made for just one or multiple ones of the disinfecting lamp 12, providing excellent expandability and flexibility of variation.

I claim:

1. An expandable disinfecting device, comprising:
a main body, which comprises a U-shaped base board, the U-shaped base board being formed with an opening, the U-shaped base board being extended from each of two sides thereof to form a side board; and
at least one disinfecting lamp, which is disposed on the side boards on the two sides of the main body, the disinfecting lamp comprising a light emission unit, a detection unit, a control unit, and a power source unit.

2. The expandable disinfecting device according to claim 1, wherein the side boards are provided with a curved edge.

3. The expandable disinfecting device according to claim 1, wherein the side boards are provided with a power accommodation trough.

4. The expandable disinfecting device according to claim 1, wherein the disinfecting lamp comprises a receiver unit, the receiver unit being a wireless signal receiver.

5. The expandable disinfecting device according to claim 1, wherein the side boards on the two sides of the U-shaped base board are arranged parallel.
